(19) United States
(12) Patent Application Publication
(10) Pub. No.: US 2006/0189949 A1
(43) Pub. Date: Aug. 24, 2006

(54) Apparatus for patients of erectile dysfunction

(76) Inventors: Kazuyuki Tomosada, Tokyo (JP); Takehisa Iwai, Tokyo (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 11/098,527

(22) Filed: Apr. 5, 2005

(30) Foreign Application Priority Data
Feb. 21, 2005 (JP) 2005-044859

(51) Int. Cl. A61F 13/00

(52) U.S. Cl. 604/304

(57) ABSTRACT

The invention provides an apparatus for patients of erectile dysfunction, which is mounted to a flaccid or semi-flaccid penis to place it in an erect state and enables the user to get through sexual intercourse as is substantially the case with a sound man. The apparatus includes an external device 10 comprising a cylindrical base 101 and a head 102 molded integrally of silicone rubber. The head 102 has a streamlined spindle-like, and has a maximum diameter larger than that of the base. The head 102 also includes an opening 105 in its top portion for discharge of emitted semen. At a position in engagement with the penis glans crown, there are provided one or more windows 1031, 1032.

### BACKGROUND OF THE INVENTION

The present invention relates generally to an apparatus for patients of erectile dysfunction, and more specifically to an external device that enables those who suffer from erectile dysfunction or impotence to sustain an erection for normal sexual intercourse.

It has recently been estimated that there are increasing numbers of impotents. So far, such patients have been treated with silicone implantation. However, silicone causes some complications upon exposure, and is still insufficient for treatment of erectile dysfunction or impotence.

Such being the case, we have filed Japanese patent application Nos. 2003-289282 (patent publication 1) and 2003-289283 (patent publication 2) to come up with apparatus for patients of erectile dysfunction, comprising an integrally molded silicone rubber device.

The external device 60 illustrated in FIGS. 23-26 of the prior art comprises a base 601 and a head 602, each in a cylindrical form, which are integrally molded of silicone rubber, as shown in FIGS. 23 and 24. The head has a streamlined spindle-like contour, and has a maximum diameter larger than the diameter of the base. The head is also provided at its top portion with an opening 605 for discharge of emitted semen. Further, the head comprises a first hollow 603 that communicates with the opening 605 and fits to the penis glans, and a second hollow 604 for making the opening 605 communicate with the first hollow 603. The prior art external device 60 has a total length of 80 to 300 mm and a maximum diameter of 30 to 45 mm; the base has a thickness of 2 to 3 mm; the second hollow has a length of 8 to 20 mm and a diameter of 8 to 15 mm; and the length from the second hollow to a solid portion of the head is 8 to 20 mm.

As shown in FIGS. 25 and 26, the shaft tool 70 of the prior art is a cylindrical shaft of the same silicone rubber, and has a given length and diameter. More specifically, the outer diameter is about 25 to 35 mm, the inner diameter is about 20 to 28 mm, and the thickness of about 2 to 3 mm.

As proposed in the art, there is also an external device having juts scattered on the inner and outer peripheries. The jut 704 on the outer periphery works as much more of an irritant to sexual intercourse while the jut 705 on the inner periphery is to ensure closer contact with a penis and fit a lot more to the external device.

However, problems with the prior art are difficulty with which it is mounted to a penis, weight acting on the penis glans, lack of direct sexual irritation, and a failure in satisfactory fixation of the shaft.

Patent Publication 1

Japanese Patent Application No. 2003-289282

Patent Publication 2

Japanese Patent Application No. 2003-289283

### SUMMARY OF THE INVENTION

A main object of the invention is to provide an apparatus for patients of erectile dysfunction, which is mounted to a penis to place it in an erect state, makes the user feel a lot more direct irritation by exposing a part of the penis glans crown, and enables the user to get through with sexual intercourse to the end. Another object of the invention is to provide an apparatus for patients of erectile dysfunction, which is reduced in total weight, and is easier to mount than ever before.

These objects are achievable by the invention embodied as follows.

(1) An apparatus for patients of erectile dysfunction, characterized by comprising a cylindrical external device 10 molded integrally of a resin, wherein:

said external device 10 comprises a cylindrical base 101 and a head 102 having a streamlined spindle-like contour, and

said head 102 is provided with an opening 105 for discharge of emitted semen in a top portion and with one or more windows at a position in engagement with the penis glans crown.

(2) An apparatus for patients of erectile dysfunction, characterized by comprising a cylindrical external device 10 molded integrally of a resin, wherein:

said external device 10 comprises a cylindrical base 101 and a head 102 having a streamlined spindle-like contour, and

said head 102 is provided with an opening 105 for discharge of emitted semen in a top and further with a cylindrical tool molded integrally of a resin.

(3) The apparatus for patients of erectile dysfunction according to (1) above, wherein said external device is integrally provided with a support sheet made of a resin.

(4) The apparatus for patients of erectile dysfunction according to (3) above, wherein said resin is an integrally molded silicone rubber.

(5) The apparatus for patients of erectile dysfunction according to (4) above, wherein said support sheet comprises an upper portion of silicone rubber having a Shore A hardness (ASTM-D412) of 35±5 and a lower portion of silicone rubber having a Shore A hardness (ASTM-D412) of 80±5.

(6) The apparatus for patients of erectile dysfunction according to any one of (3) to (5) above, wherein at a position of said support sheet corresponding to the penis glans crown, there is provided a window in association with the window provided in the top portion of said external device.

(7) The apparatus for patients of erectile dysfunction according to any one of (3) to (6) above, wherein said support sheet 20 is integrally provided on an inner periphery of an inner end portion 103 of said external device, and includes a top end portion and a base end portion wherein said base end portion includes two support sheet members 2011 and 2012.

(8) The apparatus for patients of erectile dysfunction according to any one of (3) to (7) above, wherein a top edge portion 203 of the support sheet 20 is sealed up at a topmost end thereof and integrally provided with said inner end portion 103 of said external device, wherein said top edge portion 203 extends beyond the position corresponding to the penis glans crown.

(9) The apparatus for patients of erectile dysfunction according to any one of (3) to (8) above, wherein said head 102 of said external device has an angle 0 of about 10 degrees, and said support sheet 20 has a total length of 80 to 150 mm with a top portion thereof having a maximum diameter of 25 to 40 mm.

(10) The apparatus for patients of erectile dysfunction according to any one of (3) to (9) above, wherein said external device is scattered with a plurality of juts 104 on an inner periphery of the base, or one or two or more annular ribs are provided all over said inner periphery.

(11) The apparatus for patients of erectile dysfunction according to any one of (3) to (10) above, which further comprises a cylindrical shaft reinforcement tool molded integrally of said silicone rubber.

(12) The apparatus for patients of erectile dysfunction according to (11) above, wherein said shaft reinforcement tool is a cylindrical silicone tool having an outer diameter of 25 to 40 mm and an inner diameter of 20 to 35 mm.

(13) The apparatus for patients of erectile dysfunction according to (11) or (12) above, wherein both outer and inner diameters of said shaft reinforcement tool become 1 to 5 mm thicker with distance from a top portion thereof, and said shaft reinforcement tool is configured to an R sectional shape at both ends with a plurality of juts 404 and 405 provided on both inner and outer peripheries or the inner periphery alone.

### BRIEF DESCRIPTION OF THE DRAWINGS

FIG. 1 is a left side view of the first embodiment of the external device.

FIG. 2 is a sectional view of the first embodiment of the external device.

FIG. 3 is a front view of the first embodiment of the external device.

FIG. 4 is a right side view of the first embodiment of the external device.

FIG. 5 is a rear view of the first embodiment of the external device.

FIG. 6 is a plane view of the first embodiment of the external device.

FIG. 7 is a bottom view of the first embodiment of the external device.

FIG. 8 is a left side view of the second embodiment of the external device.

FIG. 9 is a sectional view of the second embodiment of the external device.

FIG. 10 is a front view of the second embodiment of the external device.

FIG. 11 is a right side view of the second embodiment of the external device.

FIG. 12 is a rear view of the second embodiment of the external device.

FIG. 13 is a plane view of the second embodiment of the external device.

FIG. 14 is a bottom view of the second embodiment of the external device.

FIG. 15 is a front view of one embodiment of the support sheet.

FIG. 16 is a sectional view of the embodiment of the support sheet.

FIG. 17 is a plan view of the embodiment of the support sheet.

FIG. 18 is a front view of one embodiment of the winding-up rod.

FIG. 19 is a sectional view of one embodiment of the winding-up rod.

FIG. 20 is a front view of one embodiment of the shaft reinforcement tool.

FIG. 21 is an end view of the embodiment of the shaft reinforcement tool.

FIG. 22 is a front view of the pubic hair keeper sheet.

FIG. 23 is a front view of the first embodiment of the prior art external device.

FIG. 24 is a sectional view of the first embodiment of the prior art external device.

FIG. 25 is a front view of one embodiment of the prior art shaft tool.

FIG. 26 is an end view of the embodiment of the prior art shaft tool.

### EXPLANATION OF THE REFERENCE NUMERALS

10: external device,
101: base,
102: head,
103: inner portion,
1031, 1032: window,
104: protuberance or jut,
105: opening,
106: collar,
201: edge,
2011, 2012: support sheet member,
203: top portion
2031, 3032: window,
205: opening,
30: winding-up rod,
40: shaft reinforcement tool,
404: jut on the outer periphery,
405: jut on the inner periphery,
50: pubic hair keeper sheet,
60: prior art external device,
601: base,
602: head,
603: first hollow,
604: second hollow,
605: opening,
70: prior shaft tool,
704: jut on the outer periphery, and
705: jut on the inner periphery.

### EXPLANATION OF THE BEST MODE OF THE INVENTION

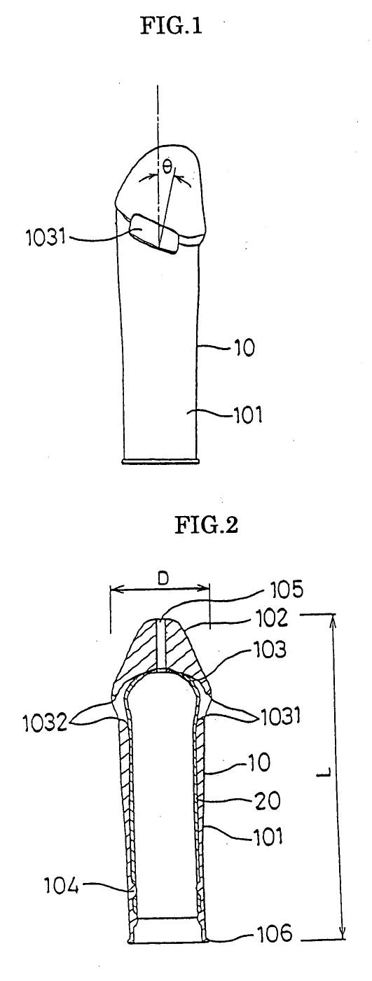

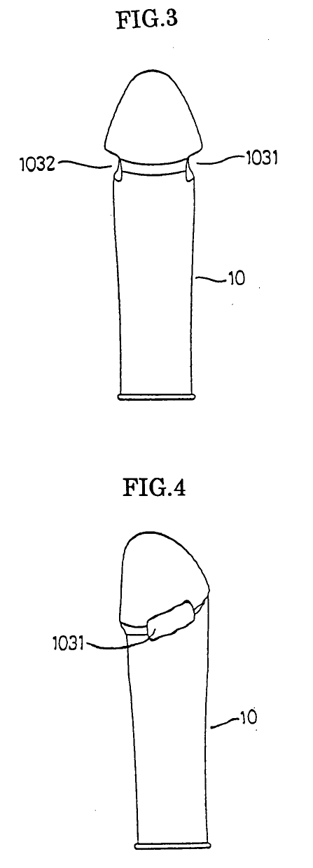

FIGS. 1, 2 and 3 are a left side view, a sectional view and a front view of the first embodiment of the invention, respectively. As shown in FIGS. 1-3, the apparatus of the invention comprises a cylindrical external device 10 generally molded integrally of silicone rubber. The device 10 has a cylindrical base 101 and a head 102. The base 101 has a length and diameter determined in such a way as to be a lot more fitted over a flaccid or semi-flaccid penis.

The head 102 is similar in shape to the penis glans in an erect state, and has a length and diameter determined in such a way to effect smooth insertion and piston movement at an angle θ of engagement of about 10 degrees with the G-spot and the uterine fundus upon insertion. In other words, the head 102 has a streamlined spindle-like contour. The tip of the head 102 is provided with an opening 105 for discharge of emitted semen.

At a position of the head 102 engaging the penis glans crown, a few windows are horizontally and vertically provided. More specifically, two windows 1031 and 1032 are horizontally provided as shown. It is noted that window size is of the order of about 5 mm to 30 mm, and that a single window could be provided in the axial direction of the head 102.

In the invention, the external device has a total length L of about 80 to 150 mm, the base has a thickness of about 1 to 5 mm, and the head 102 has a maximum diameter D of about 30 to 45 mm. The provision of the windows 1031 and 1032 in the head 102 ensures not only to maintain the feel of the user himself but also to bring the penis glans crown jutting slightly from the windows into contact with the mucosa of the partner, thereby enhancing a feel of contact. As a result, not only sexual intercourse but also oral sex such as fellatio is performable even with the external device 10 fitted over the penis. Even with the windows located, the penis glans can have sufficient stiffness due to the presence of the support sheet. On the inner periphery of the device 10 near the base, a plurality of protuberances or juts 104 are scattered. Such juts 104 may be replaced by one or two or more annular ribs provided all around the inner periphery. These are to enhance adhesion of the device to the penis or make the device fit a lot more over the penis, and prevent the apparatus of the invention from coming off as well.

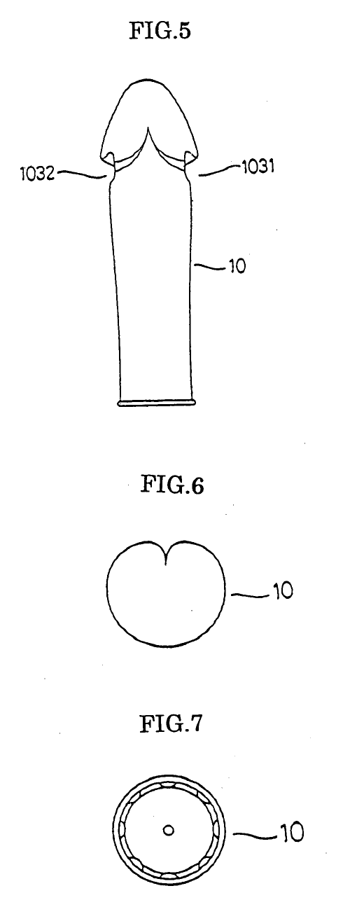

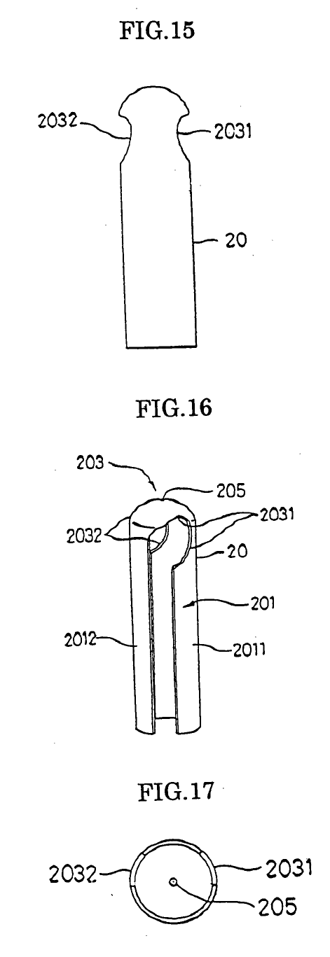

Such integral apparatus is formed of similar silicone rubber as set forth in our co-pending applications; however, it could optionally be made of various resins having similar hardness. As shown in FIGS. 15 and 16, the support sheet 20 is integrally provided on the inner periphery of an inner portion 103 of the head 102 explained with reference to FIGS. 1-7. The support sheet 20 comprises two support sheet members 2011 and 2012 and a top portion 203. The top portion 203 is sealed up at its uppermost end for integral formation with the head portion 103. An edge 201 of the support sheet 20 extends beyond a position corresponding to a portion of the exposed glans crown. At a position of the head portion 103 of the external device 10 corresponding to the opening 105, there is an opening 205 having a similar diameter. On the end side of the top 203, two axially extending support sheet members 2011 and 2012 are integrally provided in such a way as to hold the penis between them and prevent the penis from becoming flaccid halfway. At a position of the top portion 203 corresponding to the penis glans crown, there are provided windows 2031 and 2032 of similar size as mentioned above. The top portion 203 as well as each support sheet member has a thickness of about 1 to 5 mm. The support sheet has a Shore A hardness (ASTM-D412) of 5±2 as in our co-pending applications. More specifically, a portion of the support sheet above the windows 2031 and 2032 located on the top side of the support sheet members 2011 and 2012 has a Shore A hardness (ASTM-D412) of preferably 35±5, and a portion of the support sheet below the windows 2031 and 2032, that is, at the edge 201 that is below the penis and receives the penis has a Shore A hardness (ASTM-D412) of preferably 80±5. Medical silicones could be used for the silicone rubber. The silicone rubber used could comprise 80 to 95% of a silicone polymer, 5 to 20% of silica and 0.05 to 1% of titania with the rest comprising suitable amounts of phthalocyanine dyes, carbon blacks, etc.

It is noted that in the embodiment shown, the lowermost end of the base 101 is provided with a collar 106 for winding-up operation.

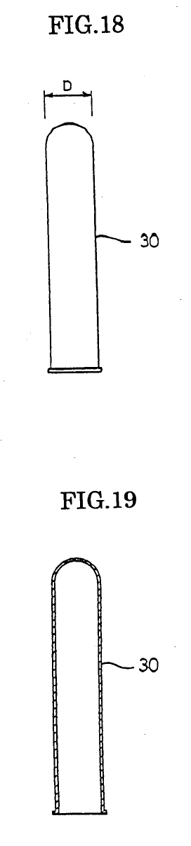

Referring to FIGS. 18 and 19, there is shown an exclusive winding-up rod 30 that is designed to facilitate winding-up operation to mount the device to a penis. For use, the external device must be in close contact and engagement with the penis, or else it will fail to exert its own effect. Satisfactory attachment of the external device to a flaccid or semi-flaccid penis often requires some force for winding-up operation. However, the use of this rod makes winding-up and attachment operation easier, and attachment time shorter as well.

While the invention has been explained with reference to silicone rubber, it is understood that suitable selection could be made from various materials.

The winding-up rod 30, for instance, may be a cylindrical rod formed of a metal such as aluminum or other materials, and have a total length of about 100 to 200 mm and a thickness of about 1 to 2 mm, with a top portion having a maximum diameter D of about 20 to 40 mm.

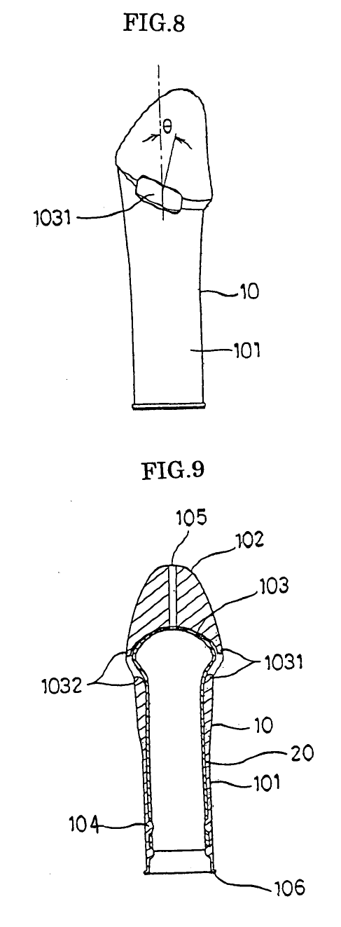

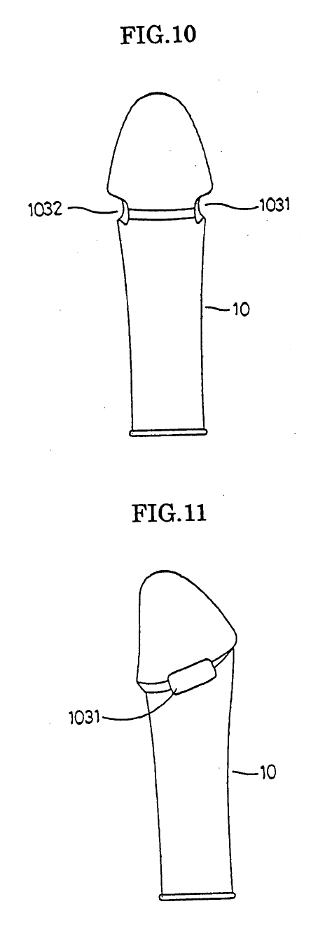

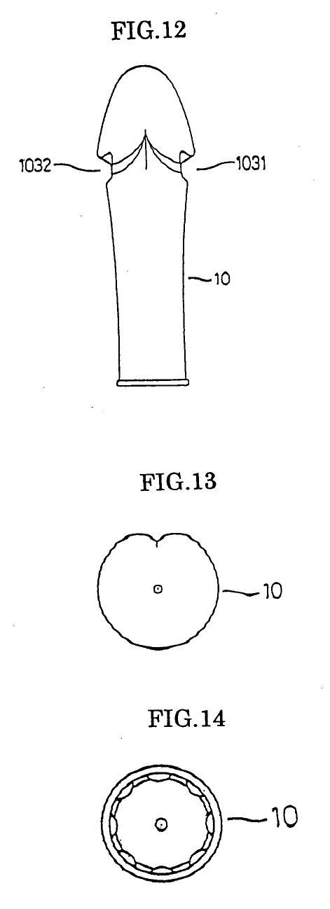

A second embodiment of the invention is depicted in FIGS. 8 to 14, and because of using much the same members as in the first embodiment, it is not explained any more. It is here noted, however, that since penis size differs from person to person, the first embodiment of FIGS. 1-7 is somewhat different in overall length and the inner and outer diameters of the head from the second embodiment of FIGS. 8-14. For instance, the second embodiment is about 10 mm, about 4 mm and about 4 mm larger than the first embodiment in terms of total length, the outer diameter of the head and the inner diameter of the head, respectively. The second embodiment is of the same thickness as the first embodiment.

A support sheet 20 is mounted on the inside of the external device 10. Typically, liquid silicone is used to bring a top portion 203 in close contact with the head portion 103 of the external device 10, followed by heating adhesion and molding. In this case, the windows 1031 and 1032 and the openings 105 and 205 must be in alignment with each other. This makes winding-up operation easier, prevents a penis from becoming flaccid halfway, and enables the external device to be mounted on even a flaccid penis. A difference in the hardness of silicone ensures to enhance the degree of close contact, thereby increasing the feel of contact of a man during sexual intercourse, and increase the strength of a shaft portion.

Referring now to FIG. 22, there is shown in a pubic hair keeper sheet 50 that is an aid to the mounting of the external device 10 or a shaft reinforcement tool 40. This sheet 50 is to prevent pubic hair from being pulled or obstructed in the apparatus. The sheet is made of an unwoven fabric of about 120 to 150 mm×about 150 to 180 mm, and includes a notch X at its center. Notch size could be about 50 to 70 mm. In use, a penis is inserted through the notch X down to its base to cover pubic hair.

Typically, the external device is mounted to a penis as follows. First, the public hair keeper sheet 50 for covering pubic hair is mounted to the base of the penis. Then, the exclusive winding-up rod 30 is inserted into the external device 10 to sufficiently wind up it from the base to the head. Once the external device has been fully wound up, the winding-up rod is removed. Then, the external device 10 is engaged with the tip of the penis glans penis and set in such a way to expose the penis glans penis crown out of the windows. Finally, the external device is wound back to the base of the penis.

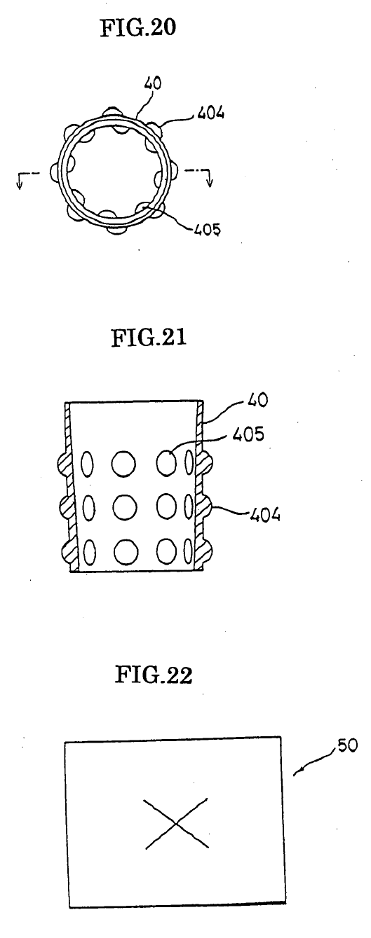

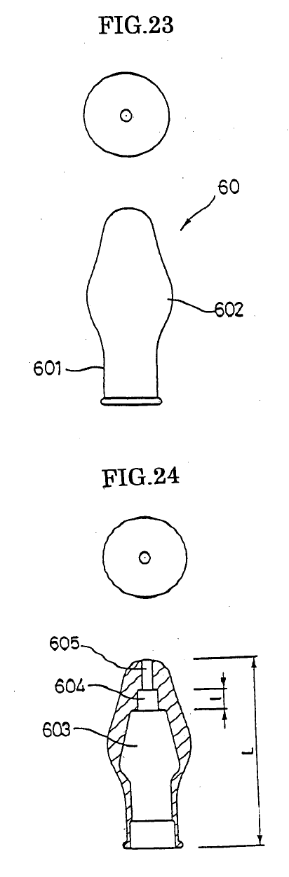

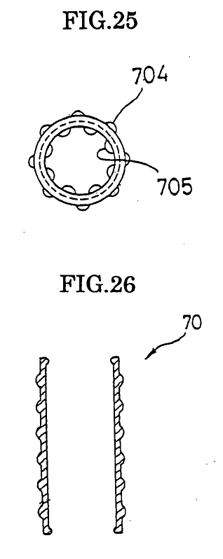

Then, the shaft reinforcement tool 40 is provided. As shown in FIGS. 20 and 21, the shaft reinforcement tool 40 is a cylindrical member made of similar silicone, having a given length and outer diameter and a similar thickness as already described. More specifically, the shaft 40 has an outer diameter of 25 to 40 mm and an inner diameter of 20 to 35 mm. To make a difference between an upper and a lower portion of the shaft, both the outer and the inner diameter of the shaft become about 1 to 5 mm thick with distance from the upper portion, and the shaft is configured to an R sectional shape at both ends for easier mounting. A plurality of protuberances or juts 404, 405 are scattered on the inner and outer peripheries of, or only the inner periphery of, the shaft. More specifically, the outer jut 404 works much more of an irritant to sexual intercourse while the inner jut 405 acts as reinforcements for close contact with a penis and fits well to the external device. Why the jut 405 on the outer periphery is in no alignment with the jut 404 on the inner periphery is to prevent the shaft from becoming thicker.

Once the external device is mounted to such a shaft reinforcement tool, the shaft reinforcement tool was forced open by inserting the index and middle fingers of both hands therein from its end, so that it is mounted to the base of the penis.

Finally, the pubic hair keeper sheet is gradually pulled out.

### ADVANTAGES AND POSSIBLE APPLICATIONS OF THE INVENTION

By the attachment of the apparatus for patients of erectile dysfunction according to the invention, a flaccid penis can be placed in an erect state, and by the provision of the windows through which the ever most sensitive penis glans crown to a male is to be exposed, it is possible for the user to enjoy sexual intercourse a lot more and, hence, make sexual intercourse to the end as is substantially the case with a sound man.

### EXAMPLE

The apparatus of the invention was fabricated of the silicone rubber to demonstrate the advantages of the invention. The external device was attached to the penis of one inventor of this invention.

More specifically, one inventor of the invention, Kazuyuki TOMOSADA was made to wear the external device on for diagnosis by another inventor, Dr. Takehisa IWAI.

The patient diagnosed as impotency was once subjected to silicone implantation. Still, impotency was not ameliorated at all due to infection or exposure of the implant. However, the attachment of the novel external device of the invention made a great contribution to amelioration in terms of the degree of satisfaction of sexual intercourse. That is, the patient's penis could be placed in an erect state without becoming flaccid halfway, thereby making substantially normal sexual intercourse at normal or back positions. Further, exposure of the penis glans crown enabled the patient to have a feel of sexual intercourse and a feel of emission as well.

What we claim is:

1. An apparatus for patients of erectile dysfunction, characterized by comprising a cylindrical external device 10 molded integrally of a resin, wherein:
said external device 10 comprises a cylindrical base 101 and a head 102 having a streamlined spindle-like contour, and
said head 102 is provided with an opening 105 for discharge of emitted semen in a top thereof and with one or more windows at a position in engagement with the penis glans crown.

2. An apparatus for patients of erectile dysfunction, characterized by comprising a cylindrical external device 10 molded integrally of a resin, wherein:
said external device 10 comprises a cylindrical base 101 and a head 102 having a streamlined spindle-like contour, and
said head 102 is provided with an opening 105 for discharge of emitted semen in a top thereof and further with a cylindrical tool molded integrally of a resin.

3. The apparatus for patients of erectile dysfunction according to claim 1, wherein said external device is integrally provided with a support sheet made of a resin.

4. The apparatus for patients of erectile dysfunction according to claim 3, wherein said resin is an integrally molded silicone rubber.

5. The apparatus for patients of erectile dysfunction according to claim 4, wherein said support sheet comprises an upper portion of silicone rubber having a Shore A hardness (ASTM-D412) of 35±5 and a lower portion of silicone rubber having a Shore A hardness (ASTM-D412) of 80±5.

6. The apparatus for patients of erectile dysfunction according to any one of claims 3 to 5, wherein at a position of said support sheet corresponding to the penis glans crown, there is provided a window in association with the window provided in the top of said external device.

7. The apparatus for patients of erectile dysfunction according to any one of claims 3 to 6, wherein said support sheet 20 is integrally provided on an inner periphery of an inner end portion 103 of said external device, and includes a top end portion and a base end portion wherein said base end portion includes two support sheet members 2011 and 2012.

8. The apparatus for patients of erectile dysfunction according to any one of claims 3 to 7, wherein a top edge portion 203 of the support sheet 20 is sealed up at a topmost end thereof and integrally provided with said inner end portion 103 of said external device, wherein said top edge portion 203 extends beyond the position corresponding to the penis glans crown.

9. The apparatus for patients of erectile dysfunction according to any one of claims 3 to 8, wherein said head 102 of said external device has an angle θ of about 10 degrees, and said support sheet 20 has a total length of 80 to 150 mm with a top portion thereof having a maximum diameter of 25 to 40 mm.

10. The apparatus for patients of erectile dysfunction according to any one of claims 3 to 9, wherein said external device is scattered with a plurality of juts 104 on an inner periphery of the base, or one or two or more annular ribs are provided all over said inner periphery.

11. The apparatus for patients of erectile dysfunction according to any one of claims 3 to 10, which further comprises a cylindrical shaft reinforcement tool molded integrally of said silicone rubber.

12. The apparatus for patients of erectile dysfunction according to claim 11, wherein said shaft reinforcement tool is a cylindrical silicone tool having an outer diameter of 25 to 40 mm and an inner diameter of 20 to 35 mm.

13. The apparatus for patients of erectile dysfunction according to claim 11 or 12, wherein both outer and inner diameters of said shaft reinforcement tool become 1 to 5 mm thicker with distance from a top portion thereof, and said shaft reinforcement tool is configured to an R sectional shape at both ends with a plurality of juts 404 and 405 provided on both inner and outer peripheries or the inner periphery alone.
